(19) United States
(12) Patent Application Publication
(10) Pub. No.: US 2022/0395679 A1
(43) Pub. Date: Dec. 15, 2022

(54) Intra-Aortic Balloon Pump

(71) Applicant: United States Government as Represented by the Department of Veterans Affairs, Washington, DC (US)

(72) Inventor: Jorge Antonio T. Gutierrez, Durham, NC (US)

(21) Appl. No.: 17/837,556

(22) Filed: Jun. 10, 2022

Related U.S. Application Data
(60) Provisional application No. 63/209,106, filed on Jun. 10, 2021.

(51) Int. Cl. A61M 60/295; A61M 60/857; A61M 60/139

(52) U.S. Cl. CPC A61M 60/295; A61M 60/857; A61M 60/139

(57) ABSTRACT

A system for improving blood flow through a circulatory system of a patient is disclosed. The patient can have a wrist. The circulatory system can have a radial artery, an ulnar artery, and a descending aorta. The system can comprise at least one inflatable bladder that is configured to be received into the descending aorta of the patient. A pump can be configured to cyclically pump a fluid into the at least one inflatable bladder to inflate the at least one inflatable bladder. A conduit can be positioned between and in fluid communication with the at least one inflatable bladder and the pump. The conduit can be configured to communicate the fluid between the pump and the at least one inflatable bladder. The conduit can have a length of at least 110 cm.

### CROSS-REFERENCE TO RELATED APPLICATIONS

This application claims priority to and the benefit of the filing date of U.S. Provisional Patent Application No. 63/209,106, filed Jun. 10, 2021, the entirety of which is hereby incorporated by reference herein.

### FIELD

This application relates to systems, apparatuses, and methods for improving blood flow through a circulatory system.

### BACKGROUND

Use of intra-aortic balloon pumps (IABPs) for hemodynamic instability secondary to cardiogenic shock is common practice. A balloon pump is typically positioned in the proximal portion of the descending aorta by way of femoral arterial access. IABPs have a balloon ranging from 30 to 50 mm that alternates from inflation and deflation according to a pumping cycle that coincides with the cardiac cycle. During contraction of the left ventricle the balloon deflates and thereby decreasing afterload aiding forward flow. During relaxation of the left ventricle, the balloon inflates to thereby enhance pressure in the ascending aorta promoting blood flow through the coronary arteries and enhancing cardiac perfusion.

Conventional IABP and left ventricular assist devices suffer from the problem that they must be inserted via the groin via an 8 French (2.667 mm outer diameter) sheath. The use of the femoral artery and larger size sheaths (greater than 6 French) are associated with increased risk of bleeding complications, such as retroperitoneal bleeding. Furthermore, the patient is required to lie flat the entire time that the balloon pump is inserted, which can be a long period of time.

### SUMMARY

Disclosed herein, in one aspect, is a system for improving blood flow through a circulatory system of a patient. The patient has a wrist and a circulatory system that has a radial artery, an ulnar artery, and a descending aorta. The system can comprise at least one inflatable bladder that is configured to be received into the descending aorta of the patient. A pump can be configured to cyclically pump a fluid into the at least one inflatable bladder to inflate the at least one inflatable bladder. A conduit can be positioned between and in fluid communication with the at least one inflatable bladder and the pump. The conduit can be configured to communicate the fluid between the pump and the at least one inflatable bladder. The conduit can have a length of at least 110 cm.

Additional advantages of the disclosed system and method will be set forth in part in the description which follows, and in part will be understood from the description, or may be learned by practice of the disclosed system and method. The advantages of the disclosed system and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed apparatus, system, and method and together with the description, serve to explain the principles of the disclosed apparatus, system, and method.

FIG. 1 illustrates a schematic diagram of an exemplary intra-aortic implantable system as disclosed herein.

FIG. 2 is an illustration of an exemplary outer tube defining fenestrations for conducting blood distally of the insertion site of the implantable system.

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “a bladder” includes one or more of such bladders, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step. As used in the specification and in the claims, the term “comprising” can include the aspects “consisting of” and “consisting essentially of.”

As used herein, “proximal” and “distal” are with reference to proximity to the heart. Thus, a proximal element is closer to the heart than a relatively distal element.

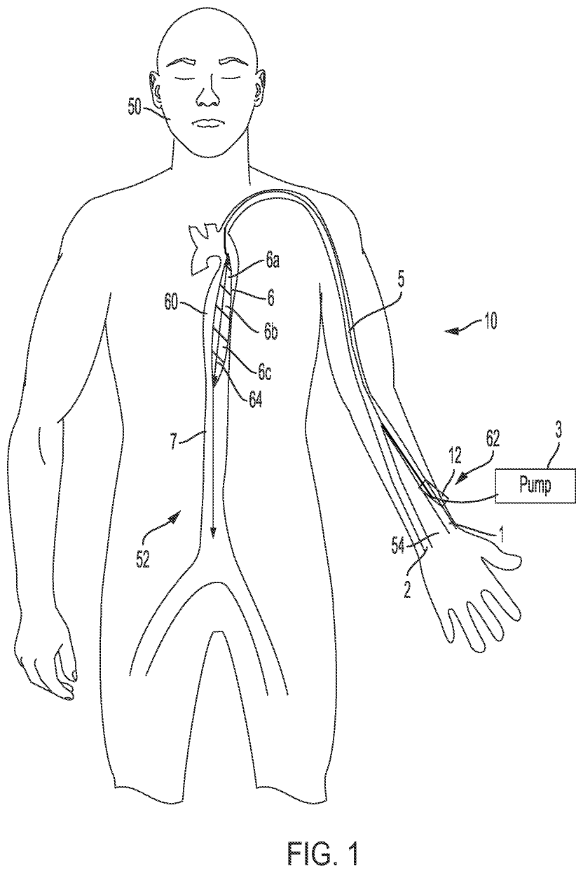

Disclosed herein, in various aspects and with reference to FIG. 1 is a system 10 for improving blood flow through a circulatory system 52 of a patient 50. The patient 50 has a wrist 54. The circulatory system 52 has a radial artery 1, an ulnar artery 2, and a descending aorta 7 having a proximal portion 60. The system 10 can comprise one or more inflatable bladders 6 that are configured to be received into the descending aorta 7 (optionally, within the proximal portion 60) of the patient 50. A pump 3 can be configured to cyclically pump a fluid into the inflatable bladder(s) 6 to inflate the inflatable bladder(s). A conduit 5 can be positioned between and in fluid communication with the at least one inflatable bladder 6 and the pump 3. The conduit 5 can be configured to communicate the fluid between the pump 3 and the at least one inflatable bladder 6. In this way, the inflatable bladder(s) 6 can be cyclically inflated to circulate blood through the circulatory system 52.

In various aspects, at least a portion of the system 10 can be insertable into the patient 50 through the radial artery 1 or the ulnar artery 2. In various optional aspects, they system 10 can be inserted into the patient 50 from an insertion site 62 proximate to the wrist 54 of the patient. Accordingly, to provide sufficient length of conduit to extend from the wrist 54 of the patient to the inflatable bladder(s) positioned within the descending aorta 7, the conduit 5 can have a length of at least 70 cm or at least 80 cm or at least 88 cm, or from about 70 cm to about 100 cm. In some aspects, the inflatable bladder can have a length (or the inflatable bladders 6 can have a collective length) of about 20 to about 30 cm, or about 22 to about 30 cm. Thus, in some aspects, the inflatable bladder(s) 6 and conduit 5 can have a combined length of at least 90 cm, at least 100 cm, or at least 110 cm. This can be in contrast to conventional IABPs that have a length of 60.5-76.5 cm and, therefore, cannot reach the descending aorta from the radial or ulnar arteries.

In various aspects, at least a portion of the system 10 can be insertable into a sheath 12 having a size of 6 French (outer diameter of 2 mm) or smaller (optionally, having a size of 5 French (outer diameter of 1.667 mm) or smaller). This can contrast with conventional IABPs that have a size of at least 8 French (outer diameter of 2.667 mm), thereby requiring use in a femoral artery that is associated with bleeding complications described herein. In some aspects, the system 10 can comprise the sheath 12.

In further aspects, it is contemplated that the system 10 can be sheathless. That is, portions of the system that enter the patient can be insertable directly into the artery without a sheath. Accordingly, the insertable portions of the system 10 (e.g., the inflatable bladders 6 and conduit 5) can have a maximum outer dimension (e.g., diameter), transverse to their dimensions of elongation, of 6 French (outer diameter of 2 mm) or smaller (optionally, having a size of 5 French (outer diameter of 1.667 mm) or smaller). In this way, it is contemplated that the system 10 can have a greater maximum dimension that is insertable into the same access site than a system 10 that is inserted into a sheath of the same outer dimension. That is, the maximum diameter of the insertable portions of the system 10 can be increased by the thickness of the sheath as compared to a system that is insertable into said sheath.

The fluid that is pumped into the inflatable bladder(s) 6 can optionally be a gas. For example, the fluid can comprise (or be) helium. In further aspects, the fluid can comprise any biocompatible fluid.

Inflation of the inflatable bladder(s) 6 can modify at least one of a length of the bladder or a diameter of the inflatable bladder(s) within the descending aorta (optionally, within the proximal portion of the descending aorta). In some aspects, the bladder(s) can be cylindrical or generally cylindrical. In this way, the bladder(s) 6 can be shaped to match the interior of the descending aorta.

In various aspects, the system can have only a single inflatable bladder 6. In further aspects, the system 10 can comprise at least two inflatable bladders 6. Optionally, the system can comprise exactly two bladders 6. In further aspects, the system 10 can comprise at least three inflatable bladders (e.g., a first bladder 6a, a second bladder 6b, and a third bladder 6c). The bladders 6 can be spaced along a longitudinal axis 64 of the system 10. Optionally, each inflatable bladder 6 can be independently inflatable. Accordingly, in various optional aspects, a respective conduit 5 can extend from the insertion site 62 to a respective inflatable bladder 6. In further aspects, two or more bladders 6 can share a single conduit 5. In these aspects, a valve can be communicatively positioned between two bladders sharing a conduit, wherein the valve can selectively be opened or closed. In an open position, the two bladders can simultaneously inflate, whereas, in a closed position, only one of the bladders can inflate. In aspects in which the system comprises three or more bladders, a respective valve can be communicatively positioned between adjacent bladders (e.g., a first valve between first and second bladders, and a second valve between second and third bladders). Optionally, said valve(s) can be set in either the open position or the closed position prior to insertion into the sheath. In further aspects, said valve can be actuatable remotely with the bladders inserted into the patient.

By providing a plurality of bladders 6, it is contemplated that the system 10 can be adapted for use with patients having different morphologies. For example, for a patient having a small descending aorta, at least one bladder can remain deflated or partially deflated. That is, only a portion of the plurality of bladders can be inflated, thereby reducing the volume of blood displaced (as compared to all bladders being inflated). For example a bladder closest to the clinician, or two or more bladders closest to the clinician, can be inflated while one or more bladders spaced farther from the clinician remain uninflated.

In some optional aspects, two or more of the inflatable bladders 6 can be inflated simultaneously. In further aspects, two or more of the inflatable bladders 6 can be inflated out of phase. For example, the pump 3 can pump the fluid into the first bladder 6a at a first cycle, and another pump (e.g., a separate pump, not shown) can pump the fluid into the second bladder 6b at a second cycle that is out of phase with the first cycle. In this way, the system 10 can create local sites of negative pressure that draw blood therein to generate blood flow through the circulatory system 52.

The sheath 12 can be insertable into the insertion site 62 in a proximal direction (toward the heart and/or torso of the patient). In further aspects, the system 10 (e.g., the one or more bladders 6 and a portion of the conduit 5) can be inserted into the patient without a sheath.

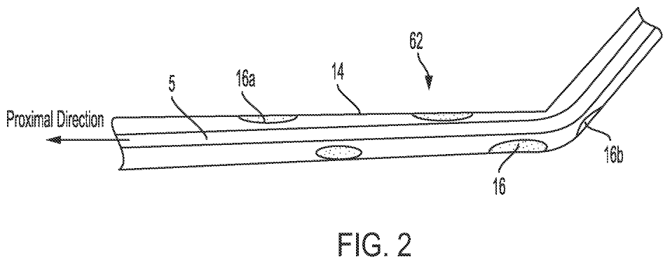

Referring to FIG. 2, optionally, the conduit 5 can be provided within an outer tube 14. The outer tube 14 can optionally define at least one fenestration 16 (optionally, a plurality of fenestrations) that is configured to permit blood flow distal of the insertion site (such that the blood moves beyond the insertion site in a direction away from the heart). That is, as fluid is pumped through the conduit to inflate the bladder(s) 6, blood can flow through one or more fenestrations 16 (e.g., openings) into an annulus between the conduit(s) 6 and the outer tube 14, distally, and then out one or more distal fenestrations 16. In this way, blood can be pumped distally beyond the system 10 and into the hand It is contemplated that the fenestrations 16 can be positioned along the tube 14 so that the fenestrations 16 are proximate to the insertion site when the bladder 6 is positioned within the aorta. In this way, as the outer tube 14 bends at the insertion site, thereby substantially blocking the inner passageway of the artery, the blood can flow into a first fenestration 16a, into the annulus between the outer tube and the conduit(s) 5, distally toward the hand, and then out another fenestration 16b that is distal of the first fenestration (and, optionally, distal of a bend in the outer tube 14). Optionally, when a plurality of fenestrations are provided, it is contemplated that at least one fenestration can be axially offset from at least one other fenestration of the plurality of fenestrations. In some aspects, each fenestration of the plurality of fenestrations can be axially offset from the other fenestrations of the plurality of fenestrations. Optionally, at least one fenestration of the plurality of fenestrations can be circumferentially offset from at least one other fenestration of the plurality of fenestrations. For example, as shown in FIG. 2, at least two groups of fenestrations 16 can be provided, with the fenestrations within each group being axially spaced but positioned at substantially the same circumferential position on the outer tube 14.

In some aspects, the system 10 can comprise instrumentation, such as, for example one or more sensors, that are positioned proximate to the bladder(s) 6. Such sensors can include a blood pressure sensor and/or a hemodynamic sensor. In this way, accurate readings of blood pressure, heart contractions, etc. can be determined at the aorta. It is contemplated that wiring can extend through the outer tube 14 for providing electrical communication to and from the sensors or other instrumentation. In some aspects, the sensors can be in communication with a controller. Optionally, the controller can further be in communication with the pump(s). In some optional aspects, the controller can be configured to control at least one parameter of the pump(s) based on feedback from the sensor(s). For example, the controller can be configured to change one or more of a frequency of pumping, a volume of fluid pumped, or a spacing between pump cycles.

According to various aspects, a method can comprise inserting the one or more bladders 6 of the system 10 into the insertion site. The insertion site 62 can extend into one of the radial artery 1 or the ulnar artery 2 proximate the wrist 54 of the patient 50. For example, the insertion site 62 can be “proximate” to the wrist 54 when positioned between the hand and the elbow of the patient 50. The bladder(s) 6 can be moved from the insertion site 62 to the proximal portion of the descending aorta. Prior to inserting the one or more bladders 6 into the insertion site 62, the sheath 12 can be inserted into the insertion site.

At least the torso of the patient can be oriented at a non-horizontal orientation during use of the system 10. For example, the torso of the patient can be oriented at least 30 degrees above horizontal (optionally, between 30 degrees and 90 degrees or between 30 degrees and 60 degrees relative to horizontal). The torso of the patient can be oriented at the non-horizontal orientation during some or all of the method described herein. For example, the torso of the patient can be oriented at the non-horizontal orientation during insertion and/or during pumping of the fluid into the inflatable bladder(s) 6.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A system for pumping blood through a circulatory system of a patient having a wrist, the circulatory system having a descending aorta, a radial artery, and an ulnar artery, the system comprising:

at least one inflatable bladder that is configured to be received into the descending aorta of the patient;

a pump that is configured to cyclically pump a fluid into the at least one inflatable bladder to inflate the at least one inflatable bladder; and

a conduit positioned between and in fluid communication with the at least one inflatable bladder and the pump, wherein the conduit is configured to communicate the fluid between the pump and the at least one inflatable bladder, wherein the conduit has a length of at least 110 cm.

Aspect 2: The system of aspect 1, wherein the conduit has a length of at least 130 cm.

Aspect 3: The system of aspect 2, wherein the conduit has a length of at least 150 cm.

Aspect 4: The system of any one of the preceding aspects, wherein the at least one inflatable bladder is insertable into a sheath having a size of 6 French (an outer diameter of 2 mm) or smaller.

Aspect 5: The system of aspect 4, wherein the at least one inflatable bladder is insertable into a sheath having a size of 5 French (an outer diameter of 1.667 mm) or smaller.

Aspect 6: The system of any one of aspects 1-5, wherein the at least one inflatable bladder comprises at least two bladders.

Aspect 7: The system of aspect 6, wherein the at least one inflatable bladder consists of two bladders.

Aspect 8: The system of aspect 6, wherein the at least one inflatable bladder consists of three bladders.

Aspect 9: The system of any one of aspects 6-8, wherein each bladder of the at least two bladders is independently inflatable.

Aspect 10: The system of any one of aspects 1-5, wherein the at least one inflatable bladder consists of a single bladder.

Aspect 11: The system of any one of the preceding aspects, further comprising a sheath that is insertable into an insertion site, wherein the insertion site extends into one of the radial artery or the ulnar artery proximate to the wrist of the patient, wherein the at least one inflatable bladder is insertable into the sheath.

Aspect 12: The system of aspect 11, wherein the sheath has a size of 6 French (an outer diameter of 2 mm) or smaller.

Aspect 13: The system of any one of the preceding claims, wherein the at least one inflatable bladder is insertable into the insertion site in a proximal direction, wherein the system further comprises an outer tube that surrounds the conduit, wherein the outer tube defines at least one fenestration that is configured to permit blood flow distal of the insertion site.

Aspect 14: A method for pumping blood through a circulatory system of a patient having a wrist, the circulatory system having a descending aorta, a radial artery, and an ulnar artery, the method comprising:

inserting at least one inflatable bladder into a sheath at an insertion site, wherein the insertion site extends into one of the radial artery or the ulnar artery proximate to the wrist of the patient; and

moving the at least one inflatable bladder from the insertion site to the descending aorta.

Aspect 15: The method of aspect 14, further comprising inserting the sheath into the insertion site prior to inserting the at least one inflatable bladder into the sheath.

Aspect 16: The method of aspect 15, wherein the sheath has a size of 6 French (an outer diameter of 2 mm) or smaller.

Aspect 17: The method of aspect 14, wherein the at least one inflatable bladder is insertable into the insertion site in a proximal direction, wherein the system further comprises an outer tube that surrounds the conduit, wherein the outer tube defines at least one fenestration that is configured to permit blood flow distal of the insertion site.

Aspect 18: The method of any one of aspects 14-17, wherein the at least one inflatable bladder has a conduit coupled thereto, wherein the at least one inflatable bladder is in fluid communication with the at least one inflatable bladder, wherein the conduit has a length of at least 110 cm.

Aspect 19: The method of aspect 18, wherein the conduit has a length of at least 130 cm.

Aspect 20: The method of aspect 19, wherein the conduit has a length of at least 150 cm.

Aspect 21: The method of any one of aspects 14-20, wherein the at least one inflatable bladder comprises at least two bladders.

Aspect 22: The method of aspect 21, wherein the at least one inflatable bladder comprises two bladders.

Aspect 23: The method of aspect 21, wherein the at least one inflatable bladder comprises three bladders.

Aspect 24: The method of any one of aspects 14-20, wherein the at least one inflatable bladder is a single bladder.

Aspect 25: The method of any one of aspects 14-24, further comprising cyclically pumping a fluid into the at least one inflatable bladder to inflate the at least one inflatable bladder.

Aspect 26: The method of aspect 25, wherein the at least one inflatable bladder comprises at a first bladder and a second bladder, wherein cyclically pumping the fluid into the at least one inflatable bladder comprises simultaneously pumping the fluid into the first bladder and the second bladder.

Aspect 27: The method of aspect 25, wherein the at least one inflatable bladder comprises at a first bladder and a second bladder, wherein cyclically pumping the fluid into the at least one inflatable bladder comprises pumping the fluid into the first bladder at a first cycle and pumping the fluid into the second bladder at a second cycle, wherein the first cycle is out of phase with the second cycle.

Aspect 28: The method of any one of aspects 25-27, wherein inflation of the at least one inflatable bladder modifies at least one of a diameter or a length of the at least one inflatable bladder within the descending aorta.

Aspect 29: The method of any one of aspects 14-28, wherein a torso of the patient is oriented in a non-horizontal orientation.

Aspect 30: The method of aspect 29, wherein the torso of the patient is oriented at least 30 degrees above horizontal.

Aspect 31: A system for pumping blood through a circulatory system of a patient having a wrist, the circulatory system having a descending aorta, a radial artery, and an ulnar artery, the system comprising:

at least one inflatable bladder that is configured to be received into the descending aorta of the patient;

a pump that is configured to cyclically pump a fluid into the at least one inflatable bladder to inflate the at least one inflatable bladder; and

a conduit positioned between and in fluid communication with the at least one inflatable bladder and the pump, wherein the conduit is configured to communicate the fluid between the pump and the at least one inflatable bladder,

wherein the at least one bladder has a transverse dimension that is less than 6 French (an outer diameter of 2 mm).

Aspect 32: The system of aspect 31, wherein the at least one bladder has a transverse dimension that is less than 5 French (an outer diameter of 1.667 mm).

Aspect 33: The system of aspect 31 or aspect 32, wherein the system does not require a sheath.

Those skilled in the art will recognize, or be able to ascertain using no more than routine experimentation, many equivalents to the specific embodiments of the method and compositions described herein. Such equivalents are intended to be encompassed by the following claims.

What is claimed is:

1. A system for pumping blood through a circulatory system of a patient having a wrist, the circulatory system having a descending aorta, a radial artery, and an ulnar artery, the system comprising:
at least one inflatable bladder that is configured to be received into the descending aorta of the patient;
a pump that is configured to cyclically pump a fluid into the at least one inflatable bladder to inflate the at least one inflatable bladder; and
a conduit positioned between and in fluid communication with the at least one inflatable bladder and the pump, wherein the conduit is configured to communicate the fluid between the pump and the at least one inflatable bladder, wherein the conduit has a length of at least 110 cm.

2. The system of claim 1, wherein the conduit has a length of at least 130 cm.

3. The system of claim 2, wherein the conduit has a length of at least 150 cm.

4. The system of claim 1, wherein the at least one inflatable bladder is insertable into a sheath having a size of 6 French (an outer diameter of 2 mm) or smaller.

5. The system of claim 4, wherein the at least one inflatable bladder is insertable into a sheath having a size of 5 French (an outer diameter of 1.667 mm) or smaller.

6. The system of claim 1, wherein the at least one inflatable bladder comprises at least two bladders.

7. The system of claim 6, wherein each bladder of the at least two bladders is independently inflatable.

8. The system of claim 1, further comprising a sheath that is insertable into an insertion site, wherein the insertion site extends into one of the radial artery or the ulnar artery proximate to the wrist of the patient, wherein the at least one inflatable bladder is insertable into the sheath.

9. The system of claim 8, wherein the sheath has a size of 6 French (an outer diameter of 2 mm) or smaller.

10. The system of claim 1, wherein the at least one inflatable bladder is insertable into the insertion site in a proximal direction, wherein the system further comprises an outer tube that surrounds the conduit, wherein the outer tube defines at least one fenestration that is configured to permit blood flow distal of the insertion site.

11. A method for pumping blood through a circulatory system of a patient having a wrist, the circulatory system having a descending aorta, a radial artery, and an ulnar artery, the method comprising:
inserting at least one inflatable bladder into a sheath at an insertion site, wherein the insertion site extends into one of the radial artery or the ulnar artery proximate to the wrist of the patient; and
moving the at least one inflatable bladder from the insertion site to the descending aorta.

12. The method of claim 11, further comprising inserting the sheath into the insertion site prior to inserting the at least one inflatable bladder into the sheath, wherein the sheath has a size of 6 French (an outer diameter of 2 mm) or smaller.

13. The method of claim 11, further comprising cyclically pumping a fluid into the at least one inflatable bladder to inflate the at least one inflatable bladder.

14. The method of claim 13, wherein the at least one inflatable bladder comprises at a first bladder and a second bladder, wherein cyclically pumping the fluid into the at least one inflatable bladder comprises simultaneously pumping the fluid into the first bladder and the second bladder.

15. The method of claim 13, wherein the at least one inflatable bladder comprises at a first bladder and a second bladder, wherein cyclically pumping the fluid into the at least one inflatable bladder comprises pumping the fluid into the first bladder at a first cycle and pumping the fluid into the second bladder at a second cycle, wherein the first cycle is out of phase with the second cycle.

16. The method of claim 11, wherein a torso of the patient is oriented in a non-horizontal orientation.

17. The method of claim 16, wherein the torso of the patient is oriented at least 30 degrees above horizontal.

18. A system for pumping blood through a circulatory system of a patient having a wrist, the circulatory system having a descending aorta, a radial artery, and an ulnar artery, the system comprising:
at least one inflatable bladder that is configured to be received into the descending aorta of the patient;
a pump that is configured to cyclically pump a fluid into the at least one inflatable bladder to inflate the at least one inflatable bladder; and
a conduit positioned between and in fluid communication with the at least one inflatable bladder and the pump, wherein the conduit is configured to communicate the fluid between the pump and the at least one inflatable bladder,
wherein the at least one bladder has a transverse dimension that is less than 6 French (an outer diameter of 2 mm).

19. The system of claim 18, wherein the at least one bladder has a transverse dimension that is less than 5 French (an outer diameter of 1.667 mm).

20. The system of claim 18, wherein the system does not comprise a sheath.
